(19) United States
(12) Patent Application Publication
(10) Pub. No.: US 2012/0316389 A1
(43) Pub. Date: Dec. 13, 2012

(54) Hygiene Protection Barrier System and Method

(76) Inventor: John Hui, Long Beach, CA (US)

(21) Appl. No.: 13/210,666

(22) Filed: Aug. 16, 2011

Related U.S. Application Data
(60) Provisional application No. 61/377,611, filed on Aug. 27, 2010.

(51) Int. Cl. A61F 6/06

(52) U.S. Cl. 600/38

(57) ABSTRACT

A protective barrier for preventing the spread of disease and the exchange of fluids between partners during oral sex is disclosed. The device, system and method may include a first portion having an area approximate to the vaginal area of a user and a second portion having a cylindrical shape with an open proximate end and a closed distal end, wherein the second portion is attached to a central portion of the first portion at the proximate end and extends into the vagina of the user.

### CROSS-REFERENCE TO RELATED APPLICATIONS

This application claims priority to U.S. Provisional Application No. 61/377,611, titled HYGIENE PROTECTION BARRIER SYSTEM AND METHOD, filed Aug. 27, 2010 and is incorporated herein as if set forth in its entirety.

### FIELD OF THE INVENTION

The present invention is directed to barrier device and, more particularly, to hygiene protection barrier device for use during oral intercourse, and a method of making and using same.

### BACKGROUND OF THE INVENTION

Although much attention has been paid to preventing the spread of disease and the exchange of fluids between partners during vaginal and anal intercourse, oral intercourse, specifically including the vaginal area of a woman, has not been adequately addressed.

Thus, there exists a need for a barrier device, system and method that prevents the spread of disease and the exchange of fluids between partners during oral sex.

### SUMMARY OF THE INVENTION

The present invention provides a barrier device and, more particularly, a hygiene protection barrier device for use during oral sex, wherein at least a portion of the vaginal area of at least one of the participants is covered by the barrier device. The barrier device may further include at least one adhesive area for securing the device to the at least one participant and may further include at least one locking mechanism for securing the device from within the at least one participant.

Thus, the invention provides a device, system and method that allows for the preventing of the spread of disease and the of exchange of fluids between partners during oral sex.

### BRIEF DESCRIPTION OF THE FIGURES

The present invention will be described herein below in conjunction with the following figures, in which like numerals represent like items, and wherein:

FIG. 1 is a plan view illustration of an embodiment of the barrier of the present invention;

FIG. 2a is an illustration of a an aspect of the present invention;

FIG. 2b is an illustration of a an aspect of the present invention;

FIG. 2c is an illustration of a an aspect of the present invention;

FIG. 2d is an illustration of a an aspect of the present invention;

FIG. 2e is an illustration of a an aspect of the present invention;

FIG. 3a is an illustration of a an aspect of the present invention; and

FIG. 3b is an illustration of a an aspect of the present invention;

FIG. 3c is an illustration of a an aspect of the present invention;

FIG. 4 is a plan view illustration of an embodiment of the barrier of the present invention; and

FIG. 5 is a side view illustration of an embodiment of the barrier of the present invention.

### DETAILED DESCRIPTION OF THE INVENTION

It is to be understood that the figures and descriptions of the present invention have been simplified to illustrate elements that are relevant for a clear understanding of the present invention, while eliminating, for the purposes of clarity, many other elements found in typical devices, systems and methods involved in physical relations. Those of ordinary skill in the art will recognize that other elements are desirable and/or required in order to implement the present invention. However, because such elements are well known in the art, and because they do not facilitate a better understanding of the present invention, a discussion of such elements is not provided herein.

The present invention provides for a protective barrier that may be used during oral sex. More specifically, the present invention provides protection for both participants during cunnilingus, herein defined to include oral sex performed within and proximate to the vaginal area.

The barrier of the present invention may consist of at least one layer of latex, or similar material suitable for skin contact and capable of preventing the transmission of viruses and germs, for example, shaped to fit and maintain substantial contact with the vaginal area. Suitable construction materials and methods thereof are disclosed in U.S. Pat. No. 5,998,540 to Lipkin, et al, issued Dec. 7, 1999 and U.S. Pat. No. 5,997,969 to Gardon, issued Dec. 7, 1999, both hereby incorporated by reference. The barrier of the present invention may also be constructed of composite elastic materials, such as multi-component materials or multilayer materials. For example, a multilayer material may have at least one elastic layer joined to at least one gatherable layer so that the gatherable layer is gathered between the locations where it is joined to the elastic layer. Such a multilayer composite elastic material may be stretched to the extent that the nonelastic material gathered between the bond locations allows the elastic material to elongate. This type of multilayer composite elastic material is disclosed, for example, by U.S. Pat. No. 4,720,415 to Vander Wielen et al., issued Jan. 19, 1988, and U.S. Pat. No. 6,096,668 to Abuto et al., issued Aug. 1, 2001, and a process for making the same is disclosed, for example, by U.S. Pat. No. 5,964,973 to Heath et al., issued Oct. 12, 1999, all of which are hereby incorporated by reference.

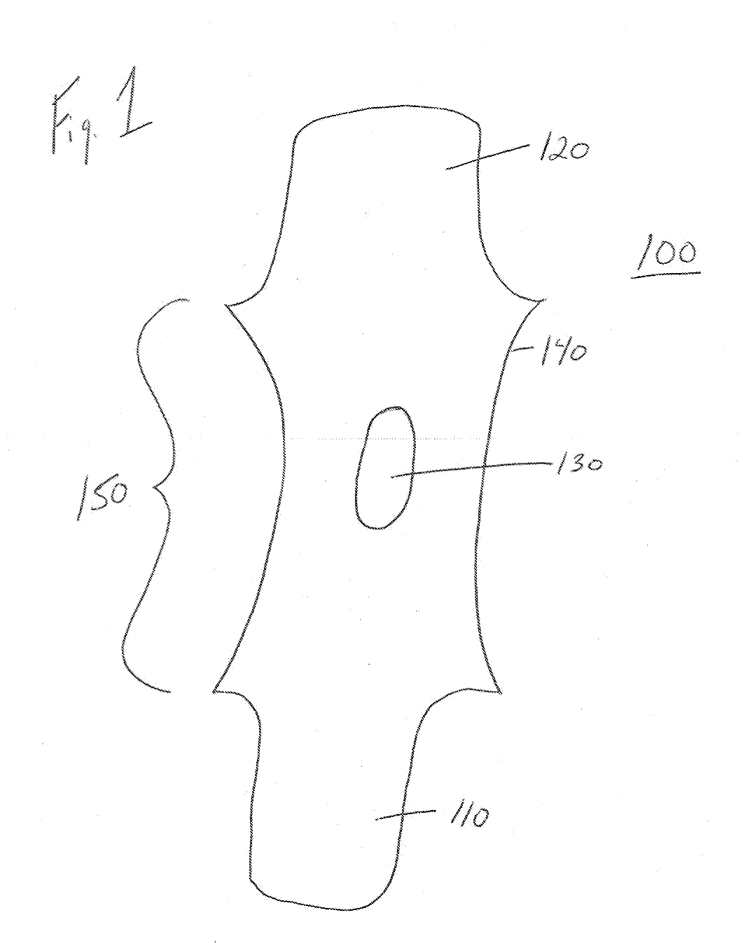

As illustrated in FIG. 1, the barrier may be die cut to include an upper flap 120, a main body 150, and a lower flap 110. The main body 150 may optionally include at least one protrusion 130 which may extend away from the main body 150. The main body 150 of the barrier 100 may be cut to conform to the natural curvature of the body where the inner thigh intersects with the vaginal area. Such a curvature cut 140 may be made uniformly within the main body 150 and/or may be obliquely cut to better accommodate variations in the size and shape of various thigh and vaginal intersections.

The main body 150 may be tall enough to substantially cover at least the pubic region of the vaginal area and at least wide enough to cover the vaginal area between the inner thighs. The main body 150, and particularly curvature, cut 140, may comprise a removable adherent for adhering to, for example, the inner thigh.

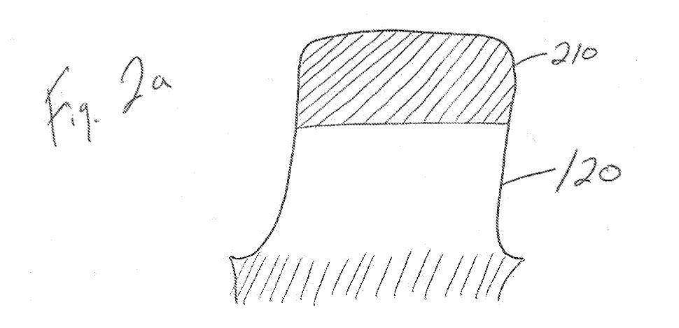

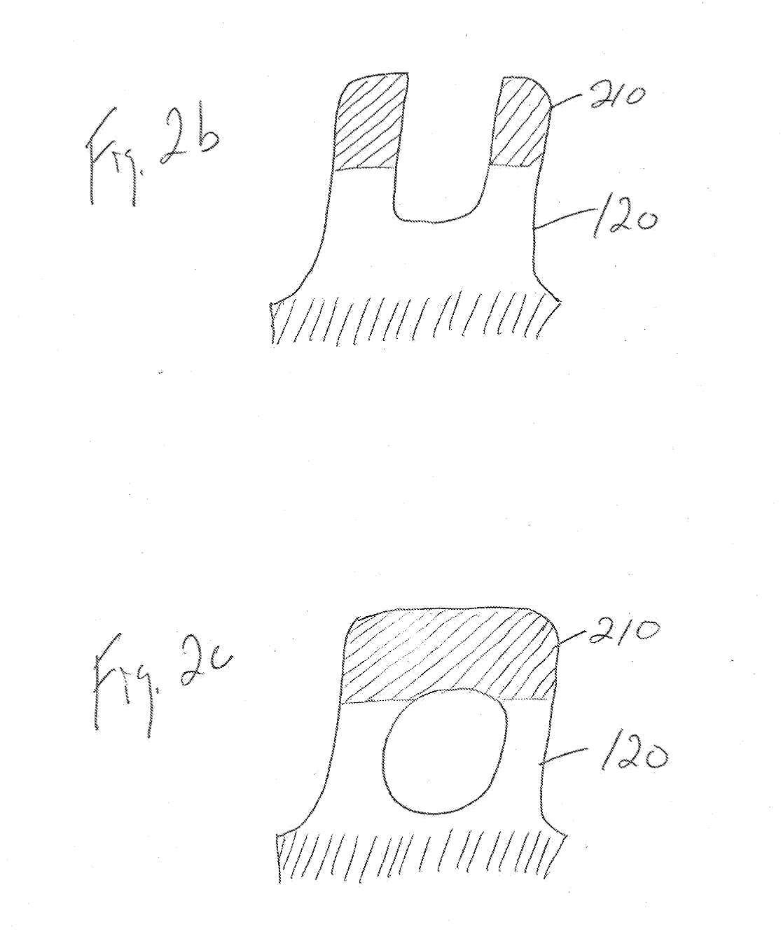

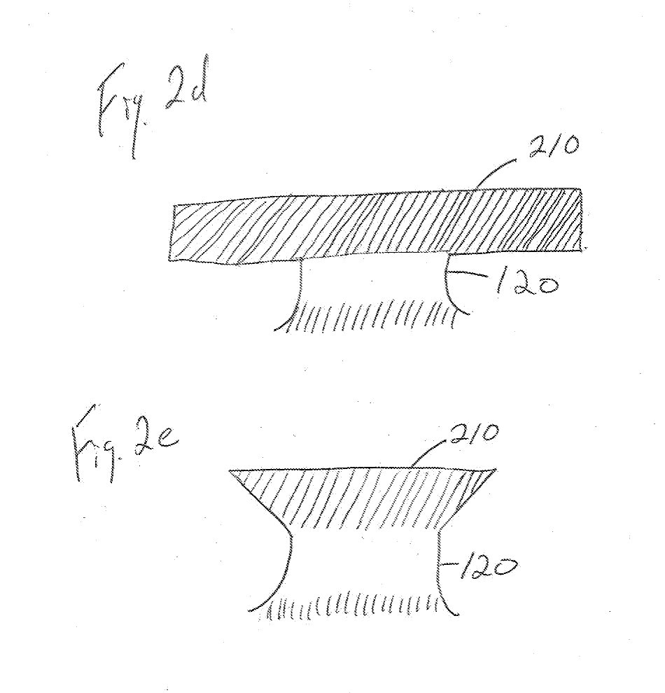

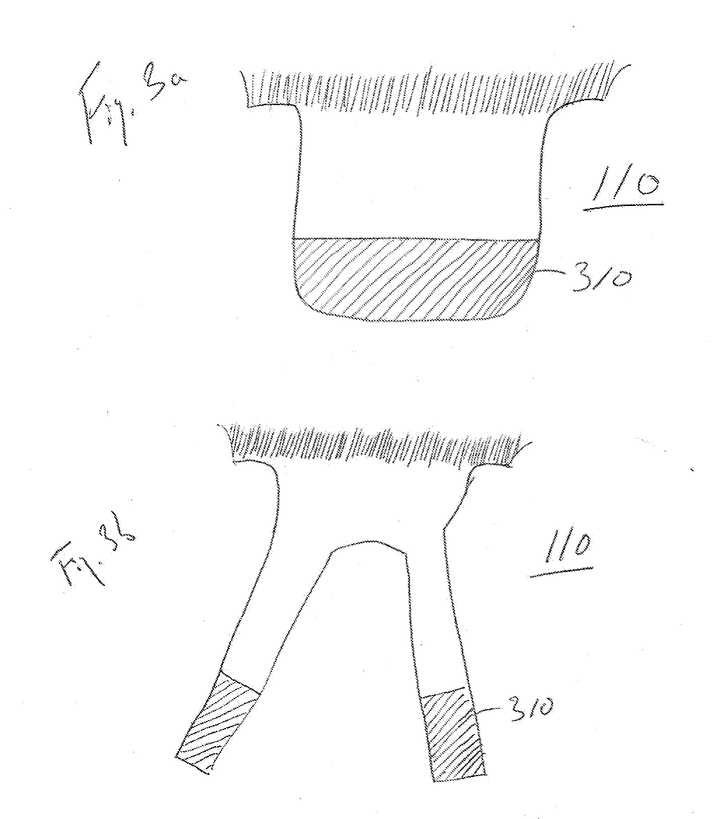

The barrier may also include an upper flap 120. The upper flap 120 may be attached to the main body 150 just above the pubic region of the vaginal area and may extend to just below the belly button, by way of non-limiting example. The upper flap 120 may be provided with at least one adhesive region 210 which may be used to secure upper flap 120 to the wearer of the barrier, as illustrated in FIGS. 2a-d.

The adhesive used may include pressure sensitive adhesives (PSA) or contact adhesives, for example, and may be applied in at least a single layer on a portion of upper flap 120 and/or curvature 140. Such an adhesive may also be a hypoallergenic suitable adhesive. Adhesive that may be used includes, but is not limited to, acrylic adhesives, and the like, e.g., a polyurethane membrane coated with a layer of an acrylic adhesive such as TEGADERM® from 3M Corporation.

Further, in an embodiment of the present invention, self-adhesive film may be used on upper flap 120 or lower flap 110. A suitable self-adhesive film is described in U.S. Pat. No. 6,663,584, to Griesbach, III, et al., issued Dec. 16, 2003, and is hereby incorporated by reference.

In an embodiment of the present invention, the upper flap 120, extends from the main body 150 to an area proximate to the wearer's belly button in a configuration similar to that illustrated in FIG. 2, and is substantially covered by at least one layer of adhesive. The relative strength of adhesive used may allow for the reduction in size of upper flap 120 which may reduce the amount of interaction with the wearer of the barrier 100.

For example, a very strong adhesive may allow the upper flap 120 to be no more than approximately 0.5 inches tall. Similarly, the amount of adhesive used may be decreased without decreasing the height of the upper flap 120. By way of further example, upper flap 120 may extend to a distance approximately halfway to the wearer's belly button and may include adhesive on only the upper half of the upper flap 120.

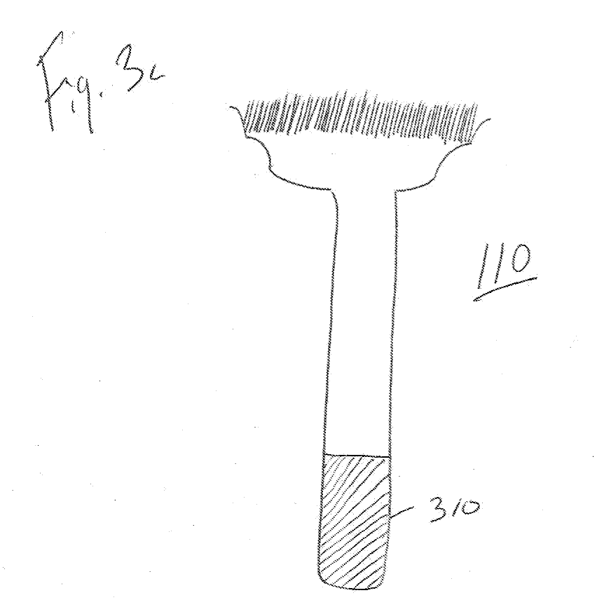

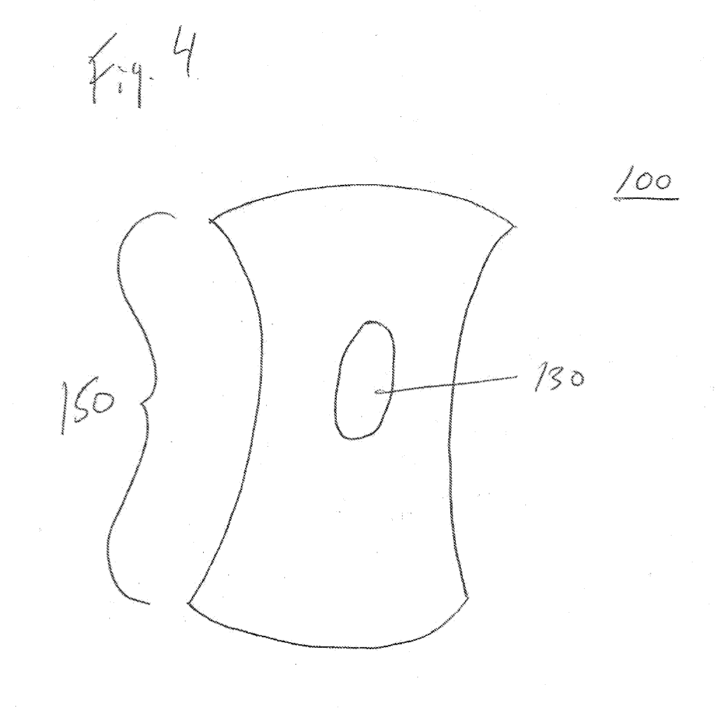

Lower flap 110 may be attached to the main body 150 and may extend to the anal region of the wearer. The properties of upper flap 120 may also be used in conjunction with lower flap 110. As illustrated in FIG. 3a, although lower flap 110 may take a shape similar to upper flap 120, lower flap 110 may have a decreased surface area given the limited size of the area between the vaginal area and anal region.

In an embodiment of the present invention as illustrated in FIG. 3b, the lower flap 110 may include at least two arms that may secure the lower portion of the main body 150. The at least two arms may, for example, extend away from the main body 150 and may terminate on opposite sides of the wearer's back-side. The ends may include adhesive and/or may be of a material which may adhere to the skin without the use of an adhesive as previously described above. The lower flap 110 may, for example, consist of a single arm as illustrated in FIG. 3c and may, for example, extend to at least the lower back of the wearer.

As illustrated in FIG. 1, the optional at least one protrusion 130 may be located proximate to the center of main body 150. Protrusion 130 may take the form of a tube that may be inserted in the vagina and may be closed at the terminal end. The protrusion 130 may be circular and/or oblong to naturally fit inside a vagina. The protrusion 130 may consist of substantially the same material as the main body 150 and may be sufficiently thin as to allow for slight pressure and movements to be felt by the wearer. The protrusion 130 may also be conical in nature which may allow for great flexibility in fit and comfort of the protrusion 130 in light of the variations between wearers.

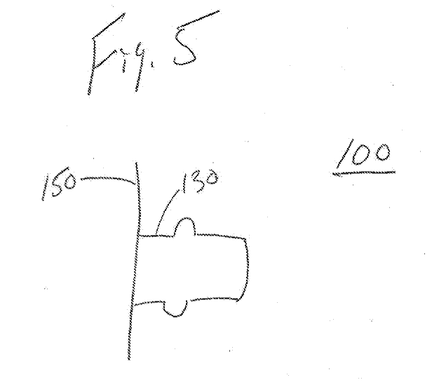

The protrusion 130 may also act as an anchor or, and may thus include an anchor or protrusion at the distal end for, the main body 150. As illustrated in FIG. 4, the barrier 100 may not include an upper flap 120 or lower flap 110. In an embodiment of the present invention, the barrier 110 consists of the main body 150 and the protrusion 130 wherein the protrusion 130 is of a sufficient length as to provide stability to main body 150 when oral sex is being administered to the wearer of barrier 100. As described above, the main body 150 may be shaped to fit between the inner thighs of the wearer and may extend to cover the whole of the vaginal region.

The protrusion 130 may include at least one protrusion remote from the surface of main body 150. Such protrusion may consist of a series of bumps, for example. As illustrated in FIG. 5, the at least one protrusion of protrusion 130 may consist of a ring formation located near the proximate end of protrusion 130. Such a ring structure may assist in anchoring the barrier 100. The at least one ring structure may be located sufficiently away from main body 150 so as to reside behind at least the labia of the wearer when fully inserted. To increase the anchoring effect of the ring structure, more than one may be included on protrusion 130.

In an embodiment of the present invention, the at least one ring structure may be a closed system to the protrusion 130 and may, for example, be filled with a material to create a more rigid structure. Materials suitable to provide rigidity may include, for example, the material used for the protrusion 130, saline, silicon, a hypo-allergenic gel and/or micro-beads. In addition to providing stability, one or more rigid ring structures may provide the wearer with stimulation. As used above, the term rigid and its like is meant to mean a greater rigidity to the protrusion 130.

The barrier 100 may be engaged by the vaginal area by placing the barrier 100 between the thighs, such as with the distal end of the protrusion 130 facing towards the wearer and with the protrusion 180 centered on the vagina. The application of the barrier 100 may apply force to push the protrusion 130 onto the vagina so as to allow the main body 150 to substantially engage the vaginal area.

In an embodiment of the present invention, the protrusion may be sufficiently long enough to accommodate, an erect penis. This may allow the barrier 100 to function as both a safety device for oral sex, but also a conventional condom without the need to switch between safety devices for both types of acts.

Thus, as would be evident to those possessing an ordinary skill in the pertinent arts, any myriad of materials may be implemented to create the prevention device of the present invention. The system of the present invention may allow for the prevention of the spread of disease and the of exchange of fluids between partners during oral intercourse and may provide similar protections during vaginal or anal intercourse.

Although the invention has been described and pictured in an exemplary form with a certain degree of particularity, it is understood that the present disclosure of the exemplary form has been made by way of example, and that numerous changes in the details of construction and combination and arrangement of parts and steps may be made without departing from the spirit and scope of the invention as set forth in the claims hereinafter.

What is claimed is:

1. A device for preventing the spread of disease, comprising:
a first portion having an area approximate to a vaginal area of a user; and
a second portion having a cylindrical shape with an open proximate end and a closed distal end;
wherein the second portion is attached to a central portion of the first portion at a circumference about the proximate end and extends into the vagina of the user.

2. The device of claim 1, wherein the second portion further comprises at least one ring structure.

3. The device of claim 2, wherein the at least one ring structure is of a suitable size and rigidity to securely fit within the vagina of the user.

4. The device of claim 1, wherein the first portion further comprises at least one flap structure.

5. The device of claim 4, wherein the at least one flap structure includes at least one adhesive.

6. The device of claim 1, wherein the at least a portion of the first portion is removably attached to at least a portion of the body of the user.

7. The device of claim 4, wherein the at least one flap is removably attached to at least one portion of the body of the user.

8. A method for protecting a user from the exchange of body fluids during oral sex using a protection device element comprising a first portion having an area approximate to the vaginal area of a user and a second portion having a cylindrical shape with an open proximate end and a closed distal end, the method comprising:
removing at least one release liner from at least one portion of the protective barrier prior to positioning the protection barrier about the user; and
removably attaching the protection device to at least one portion of the body of a user.

9. The method of claim 8, wherein removably attaching comprises mating the barrier device to the user by means of an adhesive surface on at least a portion on the barrier device.
